(19) United States
(12) Patent Application Publication
(10) Pub. No.: US 2026/0199693 A1
(43) Pub. Date: Jul. 16, 2026

(54) FEEDTHROUGH PIN CONFIGURED FOR LASER WELDING

(71) Applicant: Medtronic, Inc., Minneapolis, MN (US)

(72) Inventors: Andrew J. Ries, Lino Lakes, MN (US); Brad C. Tischendorf, Minneapolis, MN (US); Robert A. Munoz, Andover, MN (US); Hailiang Zhao, Plymouth, MN (US); Martin G. Hieb, St. Louis Park, MN (US); Andrew J. Thom, Maple Grove, MN (US)

(21) Appl. No.: 19/137,054

(22) PCT Filed: Nov. 28, 2023
(86) PCT No.: PCT/IB2023/061969

§ 371 Date: Jun. 9, 2025

Related U.S. Application Data
(60) Provisional application No. 63/476,906, filed on Dec. 22, 2022.

(51) Int. Cl. A61N 1/375; B23K 26/21; H01M 50/503

(52) U.S. Cl. CPC A61N 1/3754; B23K 26/21; H01M 50/503; H01M 2220/30

(57) ABSTRACT

The disclosure is directed to a feedthrough pin assembly and techniques related to a feedthrough pin assembly. An example of a feedthrough pin assembly includes a feedthrough pin which includes an elongated portion having a first radius, and an enlarged portion having a second radius. The first radius is smaller than the second radius. The feedthrough pin assembly may also include a spring plate having an opening with an opening radius smaller than the second radius, oriented circumferentially around a first portion of the elongated portion. The feedthrough pin assembly also includes an annulus window formed from a circumferential gap between the spring plate and the first portion of the elongated portion. The feedthrough pin assembly also includes a translucent ferrule distal to the spring plate, and circumferentially located around a second portion of the elongated portion.

This application claims the benefit of U.S. Provisional Patent Application Ser. No. 63/476,906, filed 22 Dec. 2022, the entire content of which is incorporated herein by reference.

### FIELD

This disclosure generally relates to a medical device and components, such as feedthrough pins, that may be used in a medical device.

### BACKGROUND

Some types of implantable medical devices (IMDs), such as cardiac pacemakers or implantable cardioverter defibrillators systems, may be used to provide cardiac sensing and therapy for a patient via one or more electrodes. Some IMDs include one or more feedthrough pins used for a variety of applications in implantable medical devices. Some implantable medical devices use feedthrough pins as electrical connections to terminals of a hermetically sealed battery within the implantable medical device.

The feedthrough pins may be used to electrically connect an internal part of an IMD with an external component. In particular, a feedthrough pin may be used to electrically connect the internal electrodes of a hermetically sealed battery to an external circuit or harness. Feedthrough pins that electrically connect components may be electrically insulated with nonconductive materials so as to prevent inadvertent electrical grounding of the feedthrough pin. Use of insulators around feedthrough pins may pose new challenges when the feedthrough pins are used to traverse a hermetic barrier.

### SUMMARY

In accordance with the techniques of the disclosure, an implantable medical device that utilizes a hermetically sealed battery, may be connected to an electrical harness or electrical circuitry with feedthrough pins. The feedthrough pin assemblies that may be configured to connect to the hermetically sealed battery set forth herein, may be configured to facilitate the use of laser welding techniques. Laser welding may be used to weld a feedthrough pin to a spring plate by directing laser energy onto a proximal end of a feedthrough pin. To electrically connect the feedthrough pin to other electrical components, without electrically grounding the feedthrough pin, an insulator may be disposed circumferentially around the feedthrough pin. One example of an insulator that may be oriented circumferentially around the feedthrough pin, is insulative ferrule. The ferrule may be translucent which may be an inherent property of some insulative materials. When the insulator is translucent, stray laser energy may propagate through the insulator to components distal to a proximal end of the feedthrough pin. Stray laser energy may be potentially damaging as it can result in advertent heating. In accordance with the techniques of the disclosure, stray laser energy may be absorbed or reflected by the feedthrough pin assembly, reducing the risk of damage from inadvertent heating.

In one example, a feedthrough pin assembly includes a feedthrough pin having an elongated portion with a first radius, and an enlarged portion with a second radius, the first radius being smaller than the second radius; a spring plate, having an opening with an opening radius smaller than the second radius, oriented circumferentially around the elongated portion; an annulus window, formed from a circumferential gap between the spring plate and the first portion of the elongated portion; and a translucent ferrule distal to the spring plate, and circumferentially located around a second portion of the elongated portion.

In another example, techniques for manufacturing a feedthrough pin assembly may include acquiring a feedthrough pin having an elongated portion with a first radius and an enlarged portion with a second radius, the first radius being smaller than the second radius; orienting a spring plate, having an opening with an opening radius smaller than the second radius, wherein the first radius is smaller than the second radius; orienting a spring plate, the spring plate comprising an opening with an opening radius smaller than the second radius, circumferentially around the elongated portion; forming an annulus window from a circumferential gap between the spring plate and the first portion of the elongated portion; and locating a translucent ferrule distal to the spring plate, and circumferentially around a second portion of the elongated portion.

In another example, techniques for laser welding a feedthrough pin may include orienting a feedthrough pin assembly towards a laser energy source, the feedthrough pin assembly including: a feedthrough pin including an elongated portion having a first radius, and an enlarged portion having a second radius wherein the first radius is smaller than the second radius; a spring plate, including an opening with an opening radius smaller than the second radius, oriented circumferentially around a first portion of the elongated portion; an annulus window, formed from a circumferential gap between the spring plate and the first portion of the elongated portion; and a translucent ferrule distal to the spring plate, and circumferentially located around a second portion of the elongated portion; and activating a laser energy source to emit laser energy towards the feedthrough pin assembly, wherein the enlarged portion of the feedthrough pin assembly at least one of attenuates or reflects at least a portion of the emitted laser energy.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below.

### BRIEF DESCRIPTION OF DRAWINGS

FIG. 1 is a conceptual drawing illustrating an example of an implantable medical device (IMD) operating within a patient according to various examples described in this disclosure.

FIG. 2 is a conceptual drawing illustrating an example of feed through pin assembly 200 used as a battery connection in accordance with one or more examples of this disclosure.

FIG. 3 is a conceptual drawing illustrating a technique for laser welding a feed through feedthrough pin assembly in accordance with one or more techniques of this disclosure.

FIG. 4 is a conceptual drawing illustrating an example of a feedthrough pin and ferrule configured for laser welding a feed through feedthrough pin assembly in accordance with one or more examples of this disclosure.

FIG. 5 is a conceptual drawing illustrating an example of a feedthrough pin and ferrule configured for laser welding a feed through feedthrough pin assembly in accordance with one or more examples of this disclosure.

FIG. 6 is a flow chart illustrating an example of a technique for forming feedthrough pin assembly, in accordance with one or more examples of this disclosure.

FIG. 7 is a flow chart illustrating an example of a technique for laser welding a feedthrough pin, in accordance with one or more examples of this disclosure.

FIG. 8 is a flow chart illustrating an example of a technique for manufacturing a feedthrough pin assembly, in accordance with one or more examples of this disclosure.

FIG. 9 is a flow chart illustrating an example of a technique for laser welding a feedthrough pin assembly, in accordance with one or more examples of this disclosure.

### DETAILED DESCRIPTION

An implantable medical device (IMD) may include a hermetically sealed housing and/or hermetically sealed components therein. The IMD may also include one or more feedthrough pins positioned within a portion of the hermetically sealed housing of the device. The feedthrough pins may be used to electrically connect components surrounded by a hermetically sealed housing with components or objects outside the housing. In one example, internal components of an IMD (e.g., a battery) may be hermetically sealed by a component housing (e.g., a battery housing). In other examples, the feedthrough pin may be used to electrically connect internal components (e.g., a pulse generator), hermetically sealed by a device housing, with tissue outside the IMD housing. The feedthrough pins may be partially disposed within a hermetic barrier, the hermetic barrier making up a portion of the hermetically sealed housing.

In some examples, the feedthrough pins may be partially disposed within a hermetic barrier of the housing, a proximal end extending proximal to, and a distal end extending distal to, the hermetic barrier. The distal end of the feedthrough pin and the proximal end of the feedthrough pin may be configured to electrically couple to electrical components or body tissue. In some examples, a seam may be formed between the feedthrough pin and the surrounding housing, to maintain the hermetic properties of the hermetic barrier.

In some examples, a spring plate may be oriented on a proximal surface of the hermetic barrier, having an opening for which the proximal end of the feedthrough pin extends. A seam may be configured to completely fill a portion of the opening between the spring plate and the feedthrough pin. The seam may be made from an electrically conductive material, thereby electrically connecting the spring plate to the feedthrough pin. In some examples, the feedthrough pin may be sealed to the plate with a pin-plate weld. The pin-plate weld may be formed using laser welding techniques to melt the proximal end of the feedthrough pin. Laser welding techniques may also cause an edge of the opening in the spring plate to melt, forming a weld between the melted proximal end of the feedthrough pin and the surrounding spring plate.

In some examples, a distal end of the feedthrough pin may be configured to be electrically coupled to an energy source such as a battery. For example, an energy source may be electrically coupled to the distal end of the feedthrough pin. The energy source may be surrounded by a hermitically sealed housing, and the feedthrough pin traversing an opening in a portion of the housing (e.g., hermetic barrier). Laser welding the proximal end of the feedthrough pin to the spring plate may electrically connect the energy source with electrical components electrically coupled to the spring plate through the electrical connection of the feedthrough pin.

In some examples, an energy source may be a battery used in the IMD. Some examples of battery types that may be used in IMDs include lithium ion, lithium polymer, lithium-sulfur dioxide, and lithium iodine-polyvinylpyridine. In some examples, the energy source may be a super capacitor or a wired temporary energy source. The energy source may be rechargeable. In some examples, the energy source may be a reactive single use battery, having an expected charge life that exceeds the expected operational life of the IMD.

The energy source housing may be metallic. The metallic housing may be electrically conductive. The electrically conductive energy source housing may be grounded, having a relative ground potential in reference to any other electrical potential measured in the IMD. In some cases, the metallic housing may hermetically seal the internal components of the energy source.

In one example IMDs, such as those in which the energy source is a battery, parts of the battery may include a solution within an energy reservoir, an electrode, and a battery housing. The solution may be incompatible with components outside the battery housing. The hermetically sealed battery housing may be used to prevent components from being inadvertently exposed to the solution. In particular, components outside the battery housing that are incompatible with the solution may become damaged if the solution reservoir is not hermetically sealed within the battery housing. The housing may also prevent the internal battery components from being inadvertently exposed to foreign object debris (FOD). While feedthrough pins may traverse a hermetic barrier of the hermetically sealed housing, additionally sealing techniques may be used to maintain a hermetic seal between the feedthrough pin and the barrier.

In some examples, openings in the hermetic barrier of the housing may be used to feed the feedthrough pins into the battery. A gap between the feedthrough pin and the edge of the opening may be hermetically sealed. In some examples, the gap may be sealed via laser welding techniques. In some examples, a rounded edge surface of the feedthrough pin may be circumferentially wrapped in an electrical insulator before being partially fed through the opening. The insulator may maintain electrical isolation between the conductive material of the feedthrough pin and the metallic battery housing. One example of an insulator is an insulative ferrule.

The insulative ferrule may be made of a glass or plastic with a high level of electrical resistance. The glass or plastic insulative ferrule may be translucent or transparent to optical electromagnetic energy. In particular, the glass or plastic insulative ferrule may be translucent or transparent to light energy commonly used in laser welding.

When laser welding the feedthrough pin, in some examples, laser light may be directed to impinge a proximal end of the feedthrough pin. Electromagnetic energy absorbed by the feedthrough pin may increase the temperature of the metal in the feedthrough pin at the site of the impinging light. If the temperature of the metal increases significantly, the metal may melt producing a welding material that may flow to the surrounding material. In the case of welding feedthrough pins disposed in a metallic housing of a battery, the melted feedthrough pin may weld to the surrounding metal plate (e.g., a spring plate) of the metallic battery housing.

Throughout the disclosure, reference to “electromagnetic energy,” “laser energy,” or to “laser light” should be construed to mean within the context of a welding laser generated to laser weld a medical device. The welding laser, external to a medical device, may be used in the manufacturing of the medical device, or components of the medical device. In general, such electromagnetic energy or laser light may refract or reflect off metallic, dielectric, or permeable materials resulting in stray laser light propagating in unintended directions. The stray laser light may impinge on unintended materials or components resulting in inadvertent heating. Inadvertent heating of unintended components may damage components of the medical device (e.g., IMD) during manufacturing.

A feedthrough assembly with a shouldered feedthrough pin may limit the transmission of laser energy in a feedthrough pin when laser welding the feedthrough pin (e.g., creating a pin-plate laser weld). One or more techniques are disclosed for reducing the heating of inadvertent components from stray welding laser energy during a pin-plate laser weld. The techniques of the disclosure may allow a washer flanged feedthrough pin (e.g., a feedthrough pin with an enlarged portion configured to shield laser energy) to reduce stray laser energy that projects through a gap between a feedthrough pin and a surrounding spring plate. The shielding effects of the flanged feedthrough pin may reduce inadvertent heating.

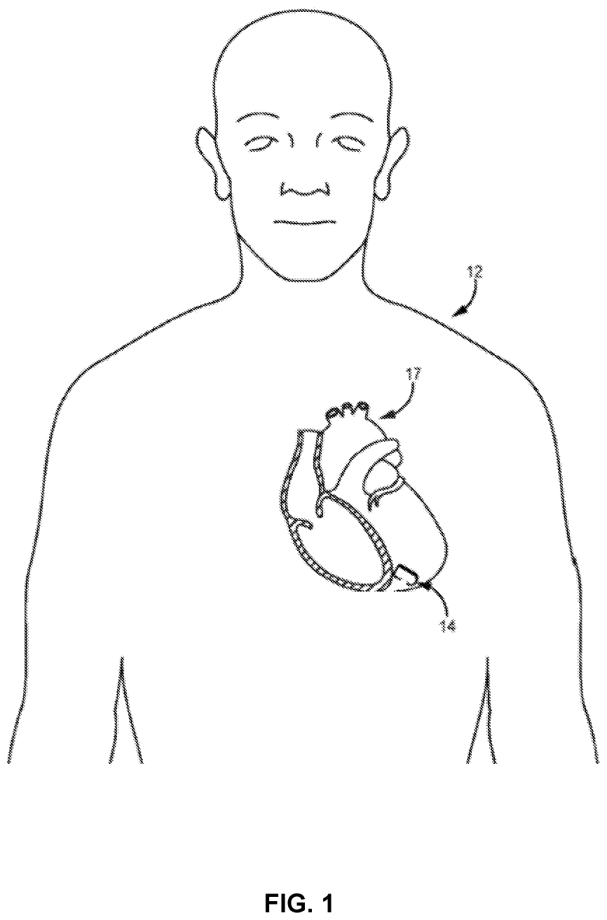

FIG. 1 is a conceptual drawing illustrating an example of IMD 14 operating within a patient 12 according to various examples described in this disclosure. The systems, devices, and techniques described in this disclosure may include examples configurations of feedthrough pins (not shown in FIG. 1) located within an IMD 14, for connecting an energy source to an IMD 14, as illustrated and described with respect to FIG. 1. For purposes of this description, knowledge of cardiovascular anatomy and functionality is presumed, and details are omitted except to the extent necessary or desirable to explain the context of the techniques of this disclosure. IMD 14, may include one or more feedthrough pins assembled with laser welding techniques. IMD 14 may be implanted at or near the site of a heart 17 of a patient 12. IMD 14 may have a hermetically sealed housing and/or include hermetically sealed component packages. The systems, devices, and techniques described herein may provide feedthrough pins that electrically connect between components within IMD 14. The systems, devices, and techniques described herein may provide feedthrough pins that electrically connect between a component with the IMD 14 and components or tissues outside IMD 14. In particular, the feedthrough pins may be disposed partially within the hermetically sealed housing of components within IMD 14 and hermetically sealed housing of IMD 14. Though described in terms of a medical device system including IMD 14, in other examples, the techniques of manufacturing techniques for feedthrough pins disclosed herein, may apply to other types of devices. Examples of other types of devices may include aerospace devices, semiconductor packages, and other devices in industries utilizing hermitic package sealing techniques.

IMD 14 may use feedthrough pins in in a variety of applications. Some of the applications may include using feedback pins as battery contacts, as signal connectors, or as other electrical connections made across a hermetically sealed barrier. In some examples, feedthrough pins may be used to connect an IMD 14 lead (not illustrated) to a main body of IMD 14. IMD 14 may be implanted outside of a thoracic cavity of patient 12 (e.g., subcutaneously in the pectoral location illustrated in FIG. 1). In other examples, IMD 14 may be positioned near the sternum near or just below the level of the heart of patient 12, e.g., at least partially within the cardiac silhouette. In other examples, IMD 14 may be implanted proximate to, attached to, or on the epicardium of heart 17, as shown in FIG. 1. In other examples, IMD 14 may be located in other locations on patient 12, including for monitoring and stimulation of the tibial nerve, sacral nerve, spinal cord, vagal nerve, deep brain stimulation, located at or near one or more organs or other locations.

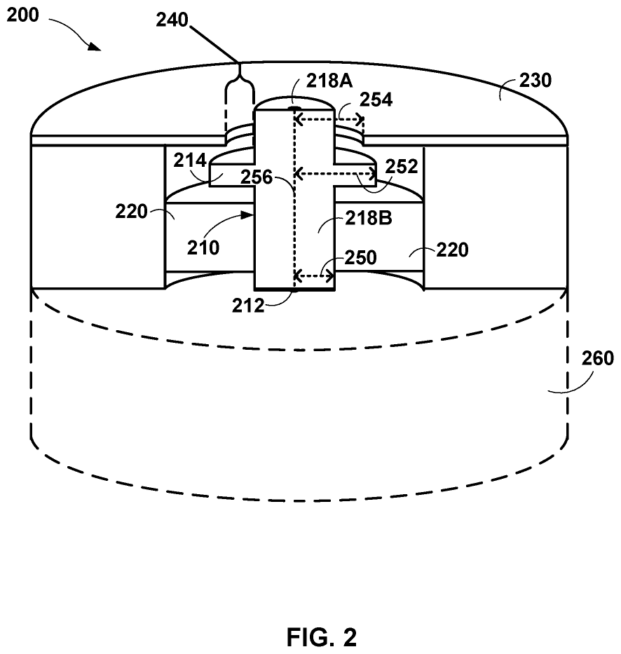

IMD 14 includes a plurality of electrodes (FIG. 2) and may be configured to sense a cardiac electrogram (EGM) and other bioelectrical signals via the plurality of electrodes. Electrodes may be integrated with the hermetically sealed housing of IMD 14. In some examples, the electrodes may be integrated with the housing using the feedthrough manufacturing techniques of this disclosure. In various examples, IMD 14 may represent a cardiac monitor, a defibrillator, a cardiac resynchronization pacer/defibrillator, a pacemaker, a ventricular assist device, an implantable pressure sensor, a neurostimulator, a glucose monitor, a drug pump, a pulse wave velocity measurement device, or any other implantable or external medical device.

For the remainder of the disclosure, a general reference to IMD 14 may refer collectively to include any examples of IMD 14, a general reference to sensor circuits may refer collectively to include any feedthrough pins used as electrical connectors.

FIG. 2 is a conceptual drawing illustrating an example of feedthrough pin assembly 200 used as a battery connection 212, in accordance with one or more examples of this disclosure. Feedthrough pin assembly 200 may include a feedthrough pin 210, partially disposed within an opening of a translucent ferrule 220. Feedthrough pin assembly 200 may also include a spring plate 230 oriented circumferentially around feedthrough pin 210.

In some examples, feedthrough pin 210 may have an elongated portion 218A-B (shown oriented vertically along a vertical axis 256 in the example of FIG. 2) and an enlarged portion 214. Elongated portion 218A-B may include a first elongated portion 218A and a second elongated portion 218B. First elongated portion 218A may be proximal to both enlarged portion 214 and second elongated portion 218B. Enlarged portion 214 may be proximal to second elongated portion 218B. First elongated portion 218A may have a first radius 250 corresponding to an outer radius of an external curved sidewall of first elongated portion 218A. Radius as it pertains to the first radius and the second radius should be interpreted as being the radius of a cross-section taken along a plane parallel to a proximal surface of spring plate 230. As used herein radius is not necessarily confined to a radius of a circular cross-section of a device, but may include a polygon, an ellipse, or other two-dimensional (2D) shape. When the cross-section is not circular, the radius is defined as half the length of the longest chord of the cross-section, extending in a straight-line from a point on the exterior edge of the shape formed by the cross-section to another point on the exterior edge of the shape formed by the cross-section. Enlarged portion 214 may have a second radius 252 corresponding to an outer radius of an external curved sidewall of enlarged portion 214. In some examples, enlarged portion 214 may be spherical. In some examples, enlarged portion 214 may have a second radius 252 defined as the distance of one half a longest chord of a cross section of the enlarged portion wherein the cross section is taken parallel to the proximal surface of spring plate 230. A chord may be defined as the distance of a straight-line segment between any two points of the cross-section of enlarged portion 214. The longest chord may be defined as a chord, of the cross-section parallel to the surface of spring plate 230 taken of enlarged portion 214, such that the chord is greater than all other chords taken of the cross-section. Second elongated portion 218B may have an outer radius 254 corresponding to an outer radius of an external curved side wall of second elongated portion 218B. In some examples, first radius 250 may be the same length as a third radius 254. Radius as it pertains to the third radius should be interpreted the longest straight radial line from a center of the feedthrough pin to a point on an internal edge, defined by annulus window 240, of spring plate 230. The center of the feedthrough pin may be vertical axis 256. Both first radius 250 and third radius 254 may be smaller than second radius 252.

In some examples, first elongated portion 218A may be partially disposed within an opening of spring plate 230. The opening within spring plate 230 may have a third radius 254 corresponding to a radius of a circular void, opening, hole, or cutout within spring plate 230. Third radius 254 of the void also corresponds to an outer radius of an annulus window 240. In some examples, third radius 254 may be smaller than second radius 252 of enlarged portion 214 and larger than first radius 250 of first elongated portion 218A. Spring plate 230 may be oriented circumferentially around first elongated portion 218A upon partially disposing first elongated portion 218A within the opening of spring plate 230.

Feedthrough pin assembly 200 may include annulus window 240, formed from a circumferential gap between spring plate 230 and first elongated portion 218A of feedthrough pin 210. A size of annulus window 240 may be configured based on first radius 250 of first elongated portion 218A and third radius 254 of the opening within spring plate 230. In some examples, annulus window 240 may be designed to have a particular size. In some examples, annulus window 240 may be the result of design margin used to produce feedthrough pin(s) 210 having a first radius 250 smaller than or equal to third radius 254 of the opening in the spring plate 230.

In some examples, a proximal end of feedthrough pin 210 may be proximal to spring plate 230, forming a portion of feedthrough pin 210 which extends above a proximal surface of spring plate 230. The portion of feedthrough pin 210 extending above spring plate 230 may be configured to weld with a laser welder. In some examples, a length of the portion of feedthrough pin 210 extending above spring plate 230 may be 0.003 inches. In some examples, feedthrough pin 210 may include a heat dissipating material. The heat dissipating material may include at least one of steel, silver, gold, or copper, or other electrically conductive material that melts in the presence of laser light. In various examples, the material of feedthrough pin 210 may include titanium, titanium alloys, niobium, platinum, platinum alloys. In some examples, the material may include titanium grade 23.

In some examples, feedthrough pin 210 may include enlarged portion 214 having second radius 252 which may be larger than third radius 254 of annulus window 240. Enlarged portion 214 may be distal annulus window 240 and proximal translucent ferrule 220. Second radius 252 may be larger than third radius 254 of second elongated portion 218B. In some examples, enlarged portion may be cylindrical, spherical, ovoidal, polyhedral, geometric or some three-dimensional (3D) asymmetric form. Second radius 252 should be interpreted as being the radius of a cross-section taken of the enlarged part 214 parallel to a proximal surface of spring plate 230. In some examples, the cross-section of enlarged part 214 parallel to the proximal surface of spring plate 230 may not be circular. When the cross-section is not circular, the second radius is defined as half the length of the longest chord of the cross-section, extending in a straight-line from a point on the exterior surface of the cross-section to another point on the exterior surface of the cross-section.

In some examples, translucent ferrule 220 may be formed circumferentially around second elongated portion 218B of elongated portion 218A-B of feedthrough pin 210. Translucent ferrule 220 may be cylindrical in form with an opening that extends from a proximal circular surface to a distal circular surface. A translucent property of translucent ferrule 220 may be the result of the translucent material from which translucent ferrule 220 is made. Translucent ferrule 220 may be made of a translucent glass, plastic, or crystal which may provide little or no attenuation to laser welding light. In various examples, translucent ferrule may include glass. In some examples, at least a portion of the translucent ferrule may be configured to at least one of attenuate or reflect at least a portion of laser welding light, such as a significant portion of laser welding light.

In some examples, spring plate 230 may include metallic material such as copper, steel, zinc, titanium, titanium alloys, niobium, platinum, platinum alloys or other metallic material that are compatible with laser welding metals. In various examples, the material of spring plate 230 may include titanium, titanium alloys, niobium, platinum, platinum alloys. In some examples, the material may include titanium grade 9 (e.g., TI GR9). Spring plate 230 may have an opening with third radius 254 which is smaller than second radius 252 of enlarged portion 214 of feedthrough pin 210. Spring plate 230 may be oriented circumferentially around first elongated portion 218A of elongated portion 218A-B of feedthrough pin 210. Spring plate 230 may be configured to receive melted filler metal created from the melting of a proximal end of first elongated portion 218A of feedthrough pin 210 by a laser. Spring plate 230 may be configured to create a thin annulus window wide enough to account for variation in feedthrough pin width and feedthrough pin placement, but thin enough to allow melted filler metal to flow between feedthrough pin 210 and spring plate 230. In some examples, spring plate 230 may be formed from a copper layer disposed as a trace layer on a printed circuit board (PCB). In some examples, spring plate 230 may have a material thickness configured to reflect or attenuate a significant portion of laser welding light. In some examples, the material thickness of spring plate 230 may be 0.004 inches. In some examples, spring plate 230 may be configured to function as an electrical contact, connecting feedthrough pin 210 to electrical components within the IMD.

In some examples, annulus window 240 may include a circumferential gap disposed between one of first elongated portion 218A and second elongated portion 218B and the opening in spring plate 230. Annulus window 240 may be configured to have third radius 254 less than second radius 252 of enlarged portion 214. Annulus window 240 may be transparent to light including laser welding light. Annulus window 240 may be configured to receive melted filler metal generated from a proximal end of feedthrough pin 210. In some examples, annulus window 240 may be an electrically insulative structure. The insulative annulus window 240 may be configured to provided electrical isolation between feedthrough pin 210 and spring plate 230, prior to laser welding. Upon laser welding the filling of annulus window 240 with filler metal generated from melted proximal end of feedthrough pin 210 may provide an electrical connection from battery 260 to electrical components of the IMD via feedthrough pin 210.

In some examples, battery 260 may be distal to a distal end of feedthrough pin 210. In some examples, battery 260 may be cylindrical, having a circular proximal surface and a circular distal surface. Battery 260 may include an electrode exposed on the circular proximal surface. Feedthrough pin 210 may comprise a distal end configured as battery connection 212 to battery 260. Feedthrough pin 210 may be configured to be electrically coupled to battery 260 via battery connection 212. The electrode exposed on the circular proximal surface may be electrically coupled to distal end of feedthrough pin 210. Upon welding the proximal end of feedthrough pin 210 to spring plate 230, battery 260 may be electrically coupled to spring plate 230 via battery connection 212 and feedthrough pin 210.

In some examples, feedthrough pin 210 may not be centrally located within the opening of spring plate 230. Annulus window 240 may be asymmetric. In some examples, enlarged portion 214 may be asymmetrical about feedthrough pin 210. Enlarged portion 214 may be oriented so as to block a line of sight between the asymmetric annulus window 240 and the proximal surface of translucent ferrule 220.

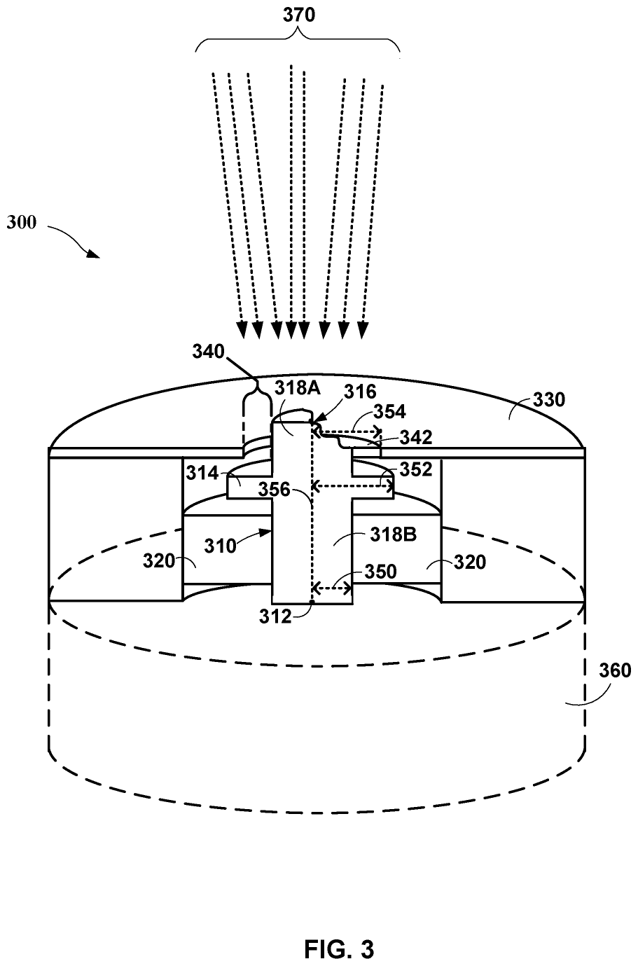

FIG. 3 is a conceptual drawing illustrating techniques for laser welding a feedthrough pin assembly 300 in accordance with one or more techniques of this disclosure. The techniques may include orienting feedthrough pin assembly 300 towards laser energy 370 generated by a laser source. Feedthrough pin assembly 300 may include a feedthrough pin 310, a spring plate 330, an annulus window 340 (similar to annulus window 240 of FIG. 2), and a translucent ferrule 320. Feedthrough pin 310 may include a first elongated portion 318A proximal an enlarged portion 314. Feedthrough pin 310 may also include a second elongated portion 318B distal enlarged portion 314 and partially disposed within translucent ferrule 320. The techniques may also include activating a laser energy source to emit laser energy 370 towards a proximal end 316 of first elongated portion 318A of feedthrough pin 310.

In some examples, first elongated portion 318A may have a radius equal in length to a first radius 350 of second elongated portion 318B. Wherein first radius 350 and the radius of first elongated portion 318A are measured from a vertical axis 356. In various examples, enlarged portion 314 of feedthrough pin 310 may be configured to reflect and/or absorb (e.g., attenuate) a substantial portion of the stray laser energy impinging upon enlarged portion 314 from the laser energy source. In various examples, enlarged portion 314 may have a second radius 352 corresponding to an outer radius of an external curved sidewall of enlarged portion 314. Second radius 352 may be configured to be larger than a third radius 354 of an opening in spring plate 330. In some examples, the radius of first elongated portion 318A may be different from first radius 350 of second elongated portion 318B. In some examples, both the radius of first elongated portion 318A and first radius 350 may smaller than second radius 352.

Welding laser may be aligned such that laser energy 370 directly impinges proximal end 316 of first elongated portion 318A of feedthrough pin 310. In some examples, welding laser energy may have a direct irradiation of 4.568345 Gigawatts per meter squared (e.g., 4568345000 W/m² ). Welding laser may cause proximal end 316, to melt, creating a partially filled 342 annulus window 340. A portion of annulus window 340, that is not filled with melted filler metal, may pass stray laser energy through annulus window 340. Passing through annulus window 340, stray laser energy, from laser energy 370, may impinge enlarged portion 314 of feedthrough pin 310, shielding the energy. Shielding stray laser energy may prevent the stray laser energy from further propagating through translucent ferrule 320. Prevention may occur when enlarged portion 314 is proximal translucent ferrule 320. In some examples, enlarged portion 314 may be distal translucent ferrule 320. When enlarged portion 314 is distal translucent ferrule 320, shielding of stray laser energy may occur after stray laser energy propagates through translucent ferrule 320.

In some examples, shielding of the stray laser energy may prevent energy from impinging a proximal end of a battery 360. Battery 360 may be connected to feedthrough pin 310 via a battery connection 312. Battery connection 312 may be made between distal end of second elongated portion 318 and a proximal surface of battery 360. Preventing stray laser energy from impinging battery 360, may limit the risk of battery damage due to over-heating from stray laser energy. In particular, limiting the amount of laser energy that impinges the proximal surface of battery 360 may limit the amount of heating battery 360 experiences from stray laser energy. In some examples, stray laser energy may impinge electronics located on or near the proximal surface of battery 360. Shielding of laser energy may also prevent heat damage to these electronics in the presence of stray laser energy.

In some examples, a portion of translucent ferrule 320 may be configured to at least one of reflect or absorb at least a portion (e.g., a significant portion) of laser welding light or laser energy. In one example, an opaque glass may be used to reflect or absorb stray laser energy. In another example, translucent ferrule 320 may be coated or plated with an optically reflective or absorptive material. The material may be configured to absorb laser energy, dissipating the laser energy as heat evenly above the battery surface.

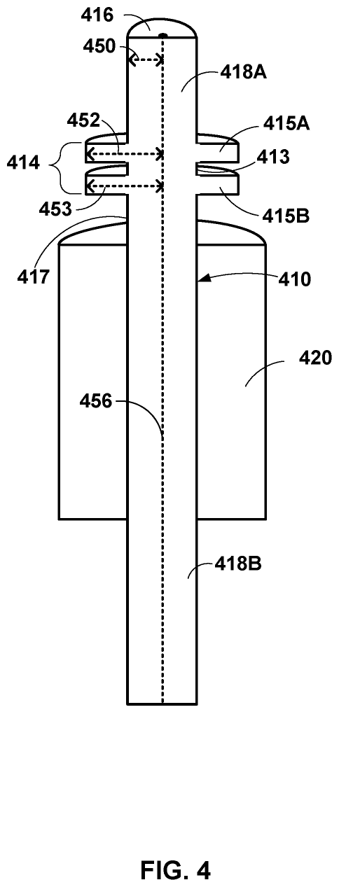

FIG. 4 is a conceptual drawing illustrating an example of a feedthrough pin and ferrule configured for laser welding a feedthrough pin assembly in accordance with one or more examples of this disclosure. Feedthrough pin 410 may include an elongated portion 418A-B and enlarged portion 414. In some examples, a first elongated portion 418A may be proximal both enlarged portion 414 and a second elongated portion 418B. Second elongated portion 418B may be distal enlarged portion 414. In various examples, second elongated portion 418B may be partially disposed within a translucent ferrule 420.

In some examples, enlarged portion 414 may include a first enlarged portion 415A and a second enlarged portion 415B. First enlarged portion 415A and second enlarged portion 415B may be separated by an intervening portion 413. In various examples, first elongated portion 418A may have a first radius 450 measured from a vertical axis 456. First radius 450 of first elongated portion 418A may be smaller than a second radius 452, also measured from vertical axis 456, of first enlarged portion 452. Second radius 452 of first enlarged portion 415A may be longer than an outer axial radius of intervening portion 413. An outer axial radius 453, measured from vertical axis 456, of second enlarged portion 415B may be larger than the outer axial radius, measured from vertical axis 456, of intervening portion 413. In some examples, outer axial radius 453 of second enlarged portion 415B may be the same length as second radius 452. In some examples, the outer axial radius of intervening portion 413 may have the same length as first radius 450 of first elongated portion 418A. In some examples, the outer axial radius of intervening portion 413 may have the same radius as an outer axial radius of second elongated portion 418B. In some examples, outer axial radius 432 of second elongated portion 418B may have the same length as first radius 450 having the same outer axial radius may be about the same with small variation in widths and thickness from manufacturing variability.

In some examples, when enlarged portion 414 is proximal translucent ferrule 420, both first enlarged portion 415A and second enlarged portion 415B may be proximal translucent ferrule 420. In some examples, when enlarged portion 414 is distal translucent ferrule 420, both first enlarged portion 415A and second enlarged portion 415B may be distal translucent ferrule 420. In some examples, first enlarged portion 415A may be proximal translucent ferrule 420, second enlarged portion 415B may be distal translucent ferrule, and intervening portion 413 may be disposed within an opening of translucent ferrule 420 (e.g., not illustrated in FIG. 4).

In some examples, both first enlarged portion 415A and second enlarged portion 415B may be configured to at least one of reflect or absorb a portion of stray laser energy (e.g., a significant portion). In some examples, one portion of either first enlarged portion 415A or second enlarged portion 415B may be configured to absorb stray while the remaining portion may be configured to reflect stray laser energy.

In some examples, a sub-portion 417 of second elongated portion 418B of feedthrough pin 410 may be proximal a proximal surface of translucent ferrule 420 and distal to enlarged portion 414. In some examples, sub-portion 417 may be oriented within intervening portion 413 (e.g., not illustrated in FIG. 4). Sub-portion 417 may form a first air gap between translucent ferrule 420 and second enlarged portion 415B, for example, for heat dissipation purposes. In some examples, sub-portion 417 may have a length corresponding to a distance between a distal surface of second enlarged portion 415B and a proximal surface of translucent ferrule 420. In some examples, the length of sub-portion 417 may be equal to or greater than 0.0005 inches but less than or equal to 0.004 inches.

In some examples, a second air gap may be formed proximal second enlarged portion 415B and distal first enlarged portion 415A. The second air gap may be formed from a difference in outer axial radius between intervening portion 413 and first enlarged portion 415A. In some examples the second air gap may be formed from a difference in outer axial radius between intervening portion 413 and second enlarged portion 415B.

In some examples, both the second air gap, between first enlarged portion 415A and second enlarged portion 415B, and the first air gap, between translucent ferrule and second enlarged portion, may be configured to improve heat dissipation. Both air gaps may be configured to improve convection cooling of first enlarged portion 415A and second enlarged portion 415B. Cooling both first enlarged portion 415A and second enlarged portion 415B using the first air gap and the second air gap, may reduce heating effects to feedthrough pin 410 from stray laser energy. In various examples, both first air gap and second air gap may be filled with at least of a variety of gases including, helium, neon, argon, or other noble gas. In various examples, first air gap and second air gap may be filled with argon gas. In particular, first enlarge portion 415A and/or second enlarged portion 415B may heat up when stray laser energy impinges on its surface.

Intervening portion 413 may have an outer axial radius equal to an outer axial radius of first elongated portion 418A of feedthrough pin 410. In some examples, the outer axial radius of intervening portion 413 may have an outer axial radius equal to an outer axial radius of second elongated portion 418B. In some examples, intervening portion 413 may extend a distance equal to the thickness of either first enlarged portion 415A or second enlarged portion 415B.

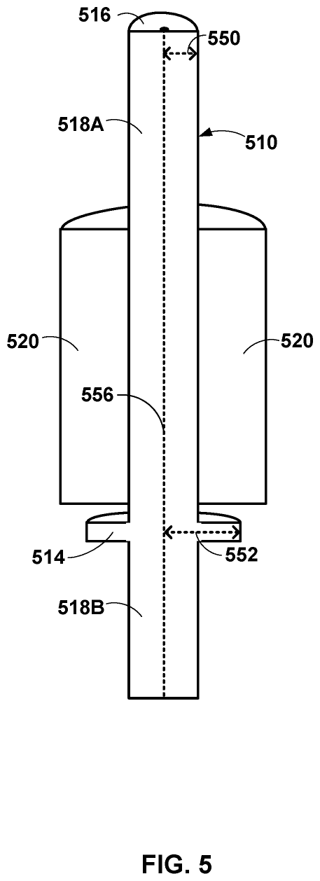

FIG. 5 is a conceptual drawing illustrating an example of a feedthrough pin 510 and translucent ferrule 520, configured for laser welding a feedthrough pin assembly in accordance with one or more examples of this disclosure. Feedthrough pin 510 may include a first elongated portion 518A proximal an enlarged portion 514. Enlarged portion 514 may be proximal second elongated portion 518B. In some examples, first elongated portion 518A may be partially disposed within an opening of translucent ferrule 520. Enlarged portion 514 may be distal translucent ferrule 520.

In some examples, first elongated portion 518A may have a first radius 550, measured from a vertical axis 556, which may be shorter than a second radius 552, also measured from vertical axis 556, of enlarged portion 514. In various examples, an outer axial radius of second elongated portion 518B, measured from vertical axis 556, may be the same length as first radius 550.

In some examples, translucent ferrule 520 may be substantially transparent to laser welding light. In particular, stray laser energy that propagates through an annulus window may enter translucent ferrule 520 from a proximal end of translucent ferrule 520. In some examples, translucent ferrule 520 may be configured to one of absorb or reflect a portion of laser light. The stray laser energy may propagate through translucent ferrule 520 and impinge on enlarged portion 514. Enlarged portion 514 may be configured to one of reflect or absorb a significant portion of stray laser energy. In some examples, a portion of translucent ferrule 520 may be configured to absorb laser energy by dissipating the energy as heat on surfaces distant from a battery or other electronic components.

Enlarged portion 514 may be configured to at least one of substantially reflect or attenuate the impinging stray laser energy propagating through translucent ferrule 520. Enlarged portion 514 may reduce the intensity of stray laser energy distal to the distal end of enlarged portion 514. Enlarged portion 514 may include at least one of copper, steel, silver, or aluminum. In some examples, enlarged portion 514 may include an electromagnetic reflective or absorptive coating to reflect or attenuate laser energy respectively.

In some examples, enlarged portion 514 may have a first outer axial radius larger than an outer axial radius of first elongated portion 518A. In some examples, enlarged portion 514 may have an outer axial radius larger than an outer axial radius of a second elongated portion 518B.

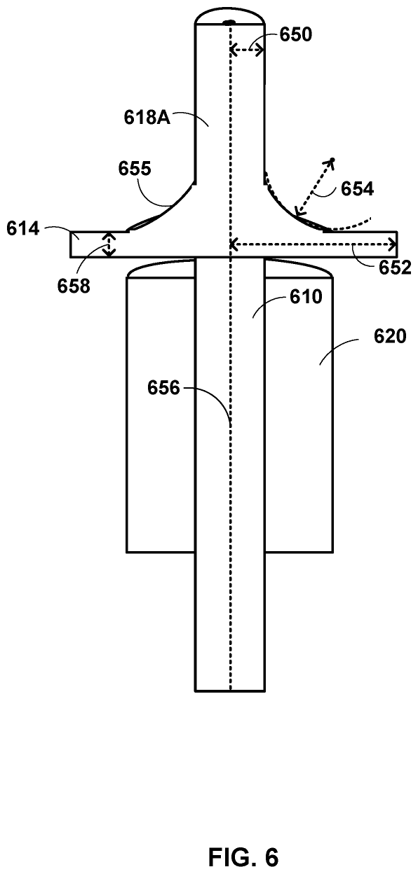

FIG. 6 is a conceptual drawing illustrating an example of a feedthrough pin 610 having a beveled reflective edge 655. In some examples, feedthrough pin 610 may include a first elongated portion 618A and a second elongated portion 618B, separated by an enlarged portion 614, configured to have beveled reflective edge 655. In some examples, first elongated portion 618A may have a first radius 650, measured as an outer axial radius from vertical axis 656. First radius 650 may be larger than a second radius 652, measured from vertical axis 656. In some examples, first radius 650 may be 0.006 inches.

In some examples, enlarged portion 614 with beveled reflective edge 655 may be designed with a bend radius 654. The beveled reflective edge 655 may be configured to reflect and/or absorb stray laser energy. In various examples, enlarged portion 614 having beveled reflective edge 655 may be proximal a translucent ferrule 620. In some examples, enlarged potion 614 with beveled reflective edge 655 may be distal translucent ferrule 620 (e.g., not illustrated in FIG. 6).

In some examples, enlarged portion 614 may have an edge thickness 658. The enlarged portion may include material from which elongated first elongated portion 618A and second elongated portion 618B were made. The edge thickness may be designed to prevent that passage of stray laser light through its material. In some examples, edge thickness 658 may be shorter than second radius 652. In some examples, edge thickness may be twice the length of bend radius 654. In some examples, edge thickness 658 may be 0.002 inches.

In some examples, beveled reflective edge 655 may have a curved surface with bend radius 654 designed to reflect light similar to a convex mirror. In some examples, beveled reflective edge 655 may be designed with a bend radius 654 to direct reflected stray laser energy toward a portion of the feedthrough pin assembly. In some examples, bend radius 654 may have a value equal to or less than 0.001 inches.

In some examples, the portion of the feedthrough pin assembly toward which the laser energy is directed may be able to absorb laser energy without damage. In some examples, the reflected stray laser energy may be reflected to an inner surface of an opening, housing the translucent ferrule and feedthrough pin. In some examples, beveled reflective edge 655 may be configured to directly reflect stray laser energy to a second reflective surface such as a corner reflector.

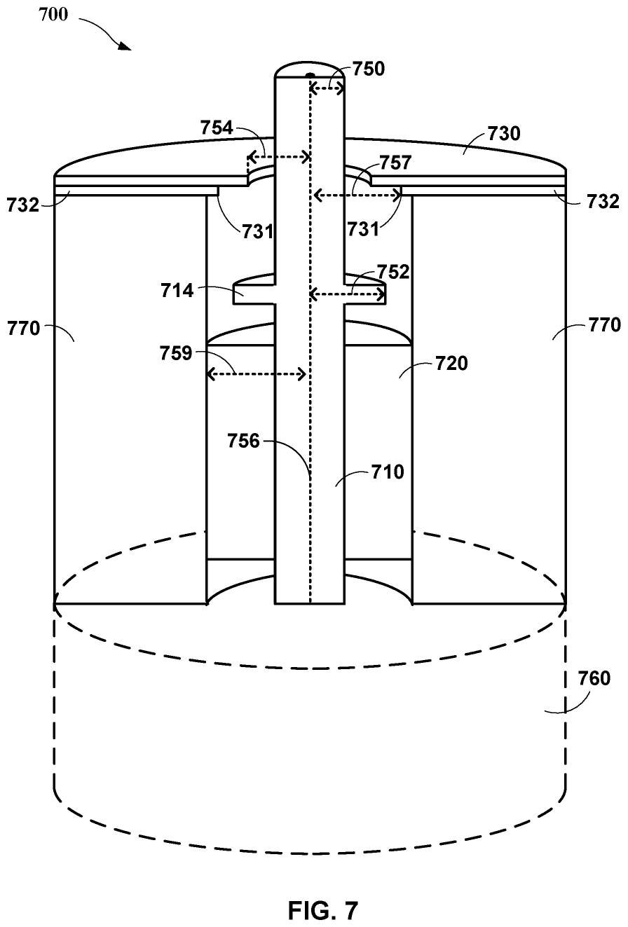

FIG. 7 is a conceptual drawing illustrating an example of a feedthrough pin assembly 700 have a corner reflector 731 configured for laser welding, in accordance with one or more examples of this disclosure. In some examples, a feedthrough pin 710 is configured to reflect stray laser energy using an enlarged portion 714, toward a corner reflector 731. Corner reflector 731 may be used to absorb and/or further reflect the laser energy.

In some examples, corner reflector 731 may be formed by placing an insulative layer 732 between a distal surface of a spring plate 730 and a proximal surface of a top cap 770. Top cap 770 may form a surrounding circumferentially around feedthrough pin 710, forming an opening in which translucent ferrule 720 and feedthrough pin 710 are situated. An inner top cap radius 759, measured from a vertical axis 756, may form the opening for inserting translucent ferrule 720 and feedthrough pin 710, prior to the placement of insulative layer 732 and spring plate 730. Corner reflector 731 may be formed from an opening in insulative layer 732, wherein the opening has an opening radius 757 shorter than inner top cap radius 759. The difference in length between opening radius 757 and inner top cap radius 759 may expose a corner of insulative layer 732, becoming corner reflector 731.

In some examples, top cap 770 may be cylindrical in shape. In various examples, top cap 770 may be formed from a metallic material such as steel, aluminum, titanium, titanium alloys, niobium, platinum, platinum alloys. In some examples, top cap 770 may be formed from material including titanium grade 1. In some examples, the opening within top cap 770 may be formed by milled out a cylindrical core from a cylindrical stock of metal.

In some examples, stray laser energy may enter a cavity, formed by the opening in the top cap, through an annulus window, formed from a gap between an opening in spring plate 730 and an outer axial surface of feedthrough pin 710. In some examples, a first radius of the elongated portion of feedthrough pin 710, measured from vertical axis 756, may be shorter than both a second radius 752, measured from vertical axis 756, of enlarged portion 714 of feedthrough pin 710, and a third radius 754, measured from vertical axis 756, of the opening in spring plate 730. The gap may be formed from the difference in length between first radius 750 of the elongated portion of feedthrough pin 710 and third radius 754 of the opening in spring plate 730.

In some examples, corner reflector 731 may be configured to absorb reflected laser energy directed to corner reflector 731 by enlarged portion 714. The absorbed energy may be dissipated as heat over a surface separated from components, fragile to high temperatures. Components located near a proximate surface of a battery 760, distal to translucent ferrule 720 may be shielded by dissipating stray laser energy with corner reflector 731.

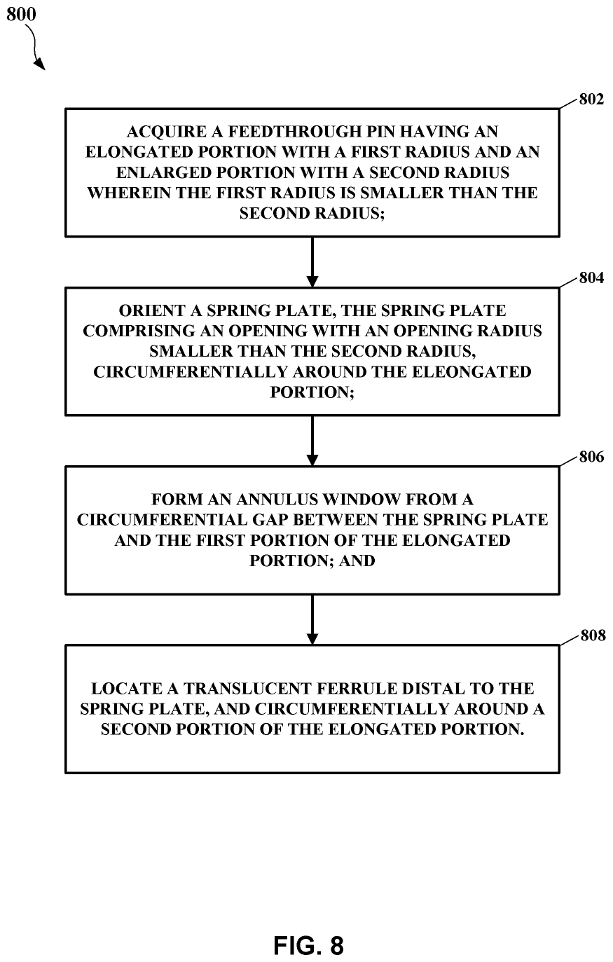

FIG. 8 is a flow chart illustrating an example of a technique 800 for manufacturing a feedthrough pin assembly, in accordance with one or more examples of this disclosure. While described with respect to feedthrough pin assembly 200 of FIG. 2, one or more of the techniques of FIG. 8 may be used with other feedthrough pin assemblies described herein. A manufacturer may acquire a feedthrough pin having an elongated portion with a first radius and an enlarged portion with a second radius wherein the first radius is smaller than the second radius (802). For example, a manufacturer may form, purchase, or otherwise acquire a feedthrough pin (e.g., feedthrough pin 210) having an elongated portion (e.g., elongated portion 218A-B) with a first radius and an enlarged portion (e.g., enlarged portion 214) with a second radius. In some examples, feedthrough pin 210 may include a distal end, configured to electrically couple to a battery. In some examples, forming the feedthrough pin (e.g., feedthrough pin 210) may include assembling, molding, or milling the feedthrough pin from stock metal. The manufacturer may mold feedthrough pin 210 by milling metal into feedthrough pin 210. When milling feedthrough pin 210, stock metal may be used in conjunction with a milling machine to form feedthrough pin 210.

A manufacturer may orient a spring plate, having an opening with an opening radius smaller than the second radius, circumferentially around the elongated portion (804). For example, a manufacturer may orient an opening in the spring plate (e.g., spring plate 230) to circumferentially surround a proximal portion (e.g., first elongated portion 218A) of a feedthrough pin (e.g., feedthrough pin 210)

A manufacturer may form an annulus window from a circumferential gap between the spring plate and the first portion of the elongated portion (806). For example, a manufacturer may form an annulus window (e.g., annulus window 240) by orienting a spring plate (e.g., spring plate 230) around a proximal end (e.g., first elongated portion 218A) creating a gap between the feedthrough pin and the spring plate, transparent to stray laser energy. In some examples, a manufacturer may form an annulus window 240 by creating a gap between a first portion (e.g., first elongated portion 218A) and a spring plate (e.g., spring plate 230), upon positioning the feedthrough pin 210 centrally within the opening of spring plate 230. The annulus window may be the result of an optically transparent gap between the feedthrough pin and the spring plate. In some examples, an air gap may not be a particular formation but an optically transparent air gap providing a line of sight to a proximal surface of translucent ferrule.

A manufacturer may locate a translucent ferrule distal to the spring plate, and circumferentially around a second portion of the elongated portion (808). For example, a manufacturer may position, locate, or orient a translucent ferrule (e.g., translucent ferrule 220) distal a spring plate (e.g., spring plate 230). The translucent ferrule (e.g., translucent ferrule 220) may be positioned, located, or oriented circumferentially around a second portion of the elongated portion (e.g., second elongated portion 218B) by feeding second elongated portion 218B partially through an opening within the translucent ferrule (e.g., translucent ferrule 220). The translucent ferrule may be electrically insulative while being electromagnetically transparent to welding laser energy. In some examples, the feedthrough pin may be centered within the opening of the translucent ferrule.

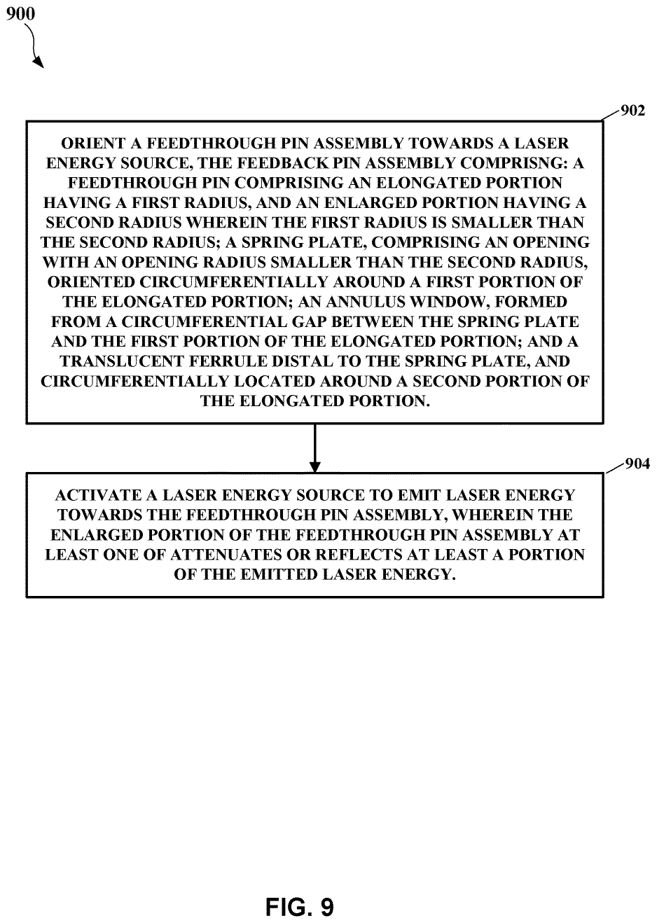

FIG. 9 is a flow chart illustrating an example of a technique 900 for laser welding a feedthrough pin assembly, in accordance with one or more examples of this disclosure. While described with respect to feedthrough pin assembly 300, one or more of the techniques of FIG. 9 may be used with other feedthrough pin assemblies described herein. In laser welding techniques for a feedthrough pin assembly, a user (e.g., manufacturer, or assembly worker) may orient a feedthrough pin assembly towards a laser energy source, the feedthrough pin assembly including a feedthrough pin including an elongated portion having a first radius, and an enlarged portion having a second radius wherein the first radius is smaller than the second radius; a spring plate, including an opening with an opening radius smaller than the second radius, oriented circumferentially around a first portion of the elongated portion; an annulus window, formed from a circumferential gap between the spring plate and the first portion of the elongated portion; and a translucent ferrule distal to the spring plate, and circumferentially located around a second portion of the elongated portion (902). In some examples, orienting a feedthrough pin assembly (e.g., feedthrough pin assembly 300) may include adjusting an orientation of a source to direct laser energy (e.g., laser energy 370) at a fixedly positioned feedthrough pin (e.g., feedthrough pin 310). In some examples, orienting a feedthrough pin assembly may include adjusting an orientation of the feedthrough assembly so as to direct laser energy from a fixedly positioned welding laser at a feedthrough pin. In some examples, directing the laser energy at a feedthrough pin may including configuring a laser spot to impinge the center of a proximal surface of the feedthrough pin. In some examples, a laser spot that impinges the proximal surface off-center may result in a greater amount and/or intensity of stray laser energy entering an annulus window.

In laser welding techniques for a feedthrough pin assembly, a user (e.g., manufacturer, or assembly worker) may activate a laser energy source to emit laser energy towards the feedthrough pin assembly, wherein the enlarged portion of the feedthrough pin assembly at least one of attenuates or reflects at least a portion of the emitted laser energy (904). Activating a laser energy source may include turning on an active welding laser. In some examples the welding lasers may include fiber laser, CO2 laser, YAG laser, gas laser, solid-state laser, and fiber laser. Different welding lasers may be used in different applications. Some applications include keyhole welding and seam welding. Activating the laser energy source may produce a laser and thereby produce the laser spot configured to impinge the proximal surface of the feedthrough pin. Activating the laser may also include maintaining the laser for a period of time while a proximal end of the feedthrough pin melts and welds to an edge of the opening of the spring plate. Activating the laser may also include deactivating the laser upon welding the feedthrough pin to the spring plate.

This disclosure includes the following non-limiting examples.

Example 1. A feedthrough pin assembly comprising: a feedthrough pin comprising an elongated portion having a first radius, and an enlarged portion having a second radius, wherein the first radius is smaller than the second radius; a spring plate having an opening with an opening radius smaller than the second radius, oriented circumferentially around a first portion of the elongated portion; an annulus window formed from a circumferential gap between the spring plate and the first portion of the elongated portion; and a translucent ferrule distal to the spring plate, and circumferentially located around a second portion of the elongated portion.

Example 2. The feedthrough pin assembly of example 1, wherein the feedthrough pin comprises a distal end, wherein the distal end is configured to be electrically coupled to a battery.

Example 3. The feedthrough pin assembly of examples 1-2, wherein the enlarged portion is positioned distal the translucent ferrule and proximal the distal end of the feedthrough pin.

Example 4. The feedthrough pin assembly of examples 1-3, wherein the enlarged portion is positioned distal the annulus window and proximal the translucent ferrule.

Example 5. The feedthrough pin assembly of examples 1-4, wherein at least a portion of the translucent ferrule is configured to at least one of attenuate or reflect at least a portion of laser welding light.

Example 6. The feedthrough pin assembly of examples 1-5, wherein the enlarged portion comprises a first enlarged portion and a second enlarged portion, the first enlarged portion and the second enlarged portion being separated by an intervening portion, wherein a radius of the first enlarged portion and a radius of the second enlarged portion are larger than a radius of the intervening portion, and wherein the radius of the intervening portion is smaller than the radius of the first enlarged portion and the radius of the second enlarged portion.

Example 7. The feedthrough pin assembly of examples 1-6, wherein the feedthrough pin comprises a heat dissipating material.

Example 8. The feedthrough pin assembly of example 7, wherein the heat dissipating material comprises at least one of steel, silver, gold, or copper.

Example 9. The feedthrough pin assembly of examples 1-8, wherein the first radius is smaller than the opening radius.

Example 10. The feedthrough pin assembly of examples 1-9, wherein first the radius is half the length of the longest chord of the cross-section, extending in a straight-line from a point on the exterior edge of the shape formed by the cross-section to another point on the exterior edge of the shape formed by the cross-section and wherein the second radius is half the length of the longest chord of the cross-section, extending in a straight-line from a point on the exterior edge of the shape formed by the cross-section to another point on the exterior edge of the shape formed by the cross-section.

Example 11. The feedthrough pin assembly of examples 1-10, wherein third radius may be defined as the distance of the longest straight-radial line from a center of the feedthrough pin to a point on an internal edge, defined by the annulus window, of the spring plate.

Example 12. A method of manufacturing a feedthrough pin assembly comprising: acquiring a feedthrough pin having an elongated portion with a first radius and an enlarged portion with a second radius wherein the first radius is smaller than the second radius; orienting a spring plate, the spring plate comprising an opening with an opening radius smaller than the second radius, circumferentially around the elongated portion; forming an annulus window from a circumferential gap between the spring plate and the first portion of the elongated portion; and locating a translucent ferrule distal to the spring plate, and circumferentially around a second portion of the elongated portion.

Example 13. The method of manufacturing a feedthrough pin assembly of example 12, wherein the feedthrough pin comprises a distal end, wherein the distal end is configured to be electrically coupled to a battery.

Example 14. The method of manufacturing a feedthrough pin assembly of examples 12-13, further comprising positioning the enlarged portion distal the translucent ferrule and proximal the distal end of the feedthrough pin.

Example 15. The method of manufacturing a feedthrough pin assembly of examples 12-14, wherein the enlarged portion is positioned distal the annulus window and proximal the translucent ferrule.

Example 16. The method of manufacturing a feedthrough pin assembly of examples 12-15, wherein the translucent ferrule is configured to at least one of attenuate or reflect at least a portion of laser welding light.

Example 17. The method of manufacturing a feedthrough pin assembly of example s 12-16, wherein the enlarged portion comprises a first enlarged portion and a second enlarged portion, the first enlarged portion and the second enlarged portion being separated by an intervening portion, wherein a radius of the first enlarged portion and a radius of the second enlarged portion are larger than a radius of the intervening portion, and wherein the radius of the intervening portion is about the same as the first radius of the feedthrough pin.

Example 18. The method of manufacturing a feedthrough pin assembly of examples 12-17, wherein feedthrough pin comprises a heat dissipating material.

Example 19. The method of manufacturing a feedthrough pin assembly of claim 18, wherein the heat dissipating material comprises at least one of steel, silver, gold, or copper.

Example 20. The method of manufacturing a feedthrough pin assembly of examples 12-19, wherein first the radius is half the length of the longest chord of the cross-section, extending in a straight-line from a point on the exterior edge of the shape formed by the cross-section to another point on the exterior edge of the shape formed by the cross-section and wherein the second radius is half the length of the longest chord of the cross-section, extending in a straight-line from a point on the exterior edge of the shape formed by the cross-section to another point on the exterior edge of the shape formed by the cross-section.

Example 21. The method of manufacturing a feedthrough pin of examples 12-20, wherein third radius may be defined as the distance of the longest straight radial line from a center of the feedthrough pin to a point on an internal edge defined by the annulus window, of the spring plate.

Example 22. A method of laser welding a feedthrough pin comprising: orienting a feedthrough pin assembly towards a laser energy source, the feedthrough pin assembly comprising: a feedthrough pin comprising an elongated portion having a first radius, and an enlarged portion having a second radius wherein the first radius is smaller than the second radius; a spring plate, comprising an opening with an opening radius smaller than the second radius, oriented circumferentially around a first portion of the elongated portion; an annulus window, formed from a circumferential gap between the spring plate and the first portion of the elongated portion; and a translucent ferrule distal to the spring plate, and circumferentially located around a second portion of the elongated portion; and activating a laser energy source to emit laser energy towards the feedthrough pin assembly, wherein the enlarged portion of the feedthrough pin assembly at least one of attenuates or reflects at least a portion of the emitted laser energy.

Example 23. The method of laser welding a feedthrough pin of example 22, wherein the feedthrough pin comprises a distal end, wherein the distal end is configured to be electrically coupled to a battery.

Example 24. The method of laser welding a feedthrough pin of examples 22-23, wherein the enlarged portion is positioned distal the translucent ferrule and proximal the distal end of the feedthrough pin.

Example 25. The method of laser welding a feedthrough pin of examples 22-24, wherein the enlarged portion is positioned distal the annulus window and proximal the translucent ferrule.

Example 26. The method of laser welding a feedthrough pin of examples 22-25, wherein at least a portion of the translucent ferrule is configured to at least one of attenuate or reflect at least a portion of laser welding light.

Example 27. The method of laser welding a feedthrough pin of examples 22-26, wherein the enlarged portion comprises a first enlarged portion and a second enlarged portion, the first enlarged portion and the second enlarged portion being separated by an intervening portion, wherein a radius of the first enlarged portion and a radius of the second enlarged portion are larger than a radius of the intervening portion, and wherein the radius of the intervening portion is about the same as the first radius of the feedthrough pin.

Example 28. The method of laser welding a feedthrough pin of examples 22-27, wherein the feedthrough pin comprises a heat dissipating material.

Example 29. The method of laser welding a feedthrough pin of examples 22-28, wherein the heat dissipating material comprises at least one of steel, silver, gold, or copper.

Example 30. The method of laser welding a feedthrough pin of examples 22-29, wherein the first radius is smaller than the opening radius.

1. A feedthrough pin assembly comprising:
a feedthrough pin comprising an elongated portion having a first radius, and an enlarged portion having a second radius, wherein the first radius is smaller than the second radius;
a spring plate having an opening with an opening radius smaller than the second radius, oriented circumferentially around a first portion of the elongated portion;
an annulus window formed from a circumferential gap between the spring plate and the first portion of the elongated portion; and
a translucent ferrule distal to the spring plate, and circumferentially located around a second portion of the elongated portion.

2. The feedthrough pin assembly of claim 1, wherein the feedthrough pin comprises a distal end, wherein the distal end is configured to be electrically coupled to a battery.

3. The feedthrough pin assembly of claim 2, wherein the enlarged portion is positioned distal the translucent ferrule and proximal the distal end of the feedthrough pin.

4. The feedthrough pin assembly of claim 1, wherein the enlarged portion is positioned distal the annulus window and proximal the translucent ferrule.

5. The feedthrough pin assembly of claim 1, wherein at least a portion of the translucent ferrule is configured to at least one of attenuate or reflect at least a portion of laser welding light.

6. The feedthrough pin assembly of claim 1, wherein the enlarged portion comprises a first enlarged portion and a second enlarged portion, the first enlarged portion and the second enlarged portion being separated by an intervening portion, wherein a radius of the first enlarged portion and a radius of the second enlarged portion are larger than a radius of the intervening portion, and wherein the radius of the intervening portion is smaller than the radius of the first enlarged portion and the radius of the second enlarged portion.

7. The feedthrough pin assembly of claim 1, wherein the feedthrough pin comprises a heat dissipating material.

8. The feedthrough pin assembly of claim 7, wherein the heat dissipating material comprises at least one of steel, silver, gold, or copper.

9. The feedthrough pin assembly of claim 1, wherein the first radius is smaller than the opening radius.

10. The feedthrough pin assembly of claim 1, wherein the first radius is half a first length of a longest chord of a cross-section of the elongated portion, and wherein the second radius is half a second length of longest chord of a cross-section of the enlarged portion.

11. The feedthrough pin assembly of claim 1, wherein third radius comprises a distance of a longest straight-radial line from a center of the feedthrough pin to a point on an internal edge, defined by the annulus window, of the spring plate.

12. A method of manufacturing a feedthrough pin assembly comprising:
acquiring a feedthrough pin having an elongated portion with a first radius and an enlarged portion with a second radius wherein the first radius is smaller than the second radius;
orienting a spring plate, the spring plate comprising an opening with an opening radius smaller than the second radius, circumferentially around the elongated portion;
forming an annulus window from a circumferential gap between the spring plate and a first portion of the elongated portion; and
locating a translucent ferrule distal to the spring plate, and circumferentially around a second portion of the elongated portion.

13. The method of manufacturing a feedthrough pin assembly of claim 12, wherein the feedthrough pin comprises a distal end, wherein the distal end is configured to be electrically coupled to a battery.

14. The method of manufacturing a feedthrough pin assembly of claim 13, further comprising positioning the enlarged portion distal the translucent ferrule and proximal the distal end of the feedthrough pin.

15. A method of laser welding a feedthrough pin comprising:
orienting a feedthrough pin assembly towards a laser energy source, the feedthrough pin assembly comprising:
a feedthrough pin comprising an elongated portion having a first radius, and an enlarged portion having a second radius wherein the first radius is smaller than the second radius;
a spring plate, comprising an opening with an opening radius smaller than the second radius, oriented circumferentially around a first portion of the elongated portion;
an annulus window, formed from a circumferential gap between the spring plate and the first portion of the elongated portion; and
a translucent ferrule distal to the spring plate, and circumferentially located around a second portion of the elongated portion; and
activating a laser energy source to emit laser energy towards the feedthrough pin assembly, wherein the enlarged portion of the feedthrough pin assembly at least one of attenuates or reflects at least a portion of the emitted laser energy.

16. The method of manufacturing a feedthrough pin assembly of claim 12, wherein the enlarged portion is positioned distal the annulus window and proximal the translucent ferrule.

17. The method of manufacturing a feedthrough pin assembly of claim 12, wherein the translucent ferrule is configured to at least one of attenuate or reflect at least a portion of laser welding light.

18. The method of manufacturing a feedthrough pin assembly of claim 12, wherein the enlarged portion comprises a first enlarged portion and a second enlarged portion, the first enlarged portion and the second enlarged portion being separated by an intervening portion, wherein a radius of the first enlarged portion and a radius of the second enlarged portion are larger than a radius of the intervening portion, and wherein the radius of the intervening portion is equal to the first radius of the feedthrough pin.

19. The method of manufacturing a feedthrough pin assembly of claim 12, wherein feedthrough pin comprises a heat dissipating material.

20. The method of manufacturing a feedthrough pin assembly of claim 19, wherein the heat dissipating material comprises at least one of steel, silver, gold, or copper.
